(19) United States
(12) Patent Application Publication
(10) Pub. No.: US 2004/0073254 A1
(43) Pub. Date: Apr. 15, 2004

(54) Suture retriever with in-line actuating handle

(76) Inventors: Jeffrey Wyman, Naples, FL (US); Robert M. Weber, Chino Hills, CA (US)

Correspondence Address: DICKSTEIN SHAPIRO MORIN & OSHINSKY LLP, 2101 L STREET NW, WASHINGTON, DC, 20037-1526, US

(21) Appl. No.: 10/679,438

(22) Filed: Oct. 7, 2003

Related U.S. Application Data
(60) Provisional application No. 60/416,536, filed on Oct. 8, 2002.

(51) Int. Cl. A61B 17/28

(52) U.S. Cl. 606/205

(57) ABSTRACT

An instrument for retrieving suture within a patient has a shaft with proximal and distal ends. The distal end terminates in an articulating mechanism, such as a jaw that captures suture slidably within an opening disposed toward the instrument tip. An actuating mechanism disposed on a handle proximal to the shaft opens and closes the jaw. The handle is adapted to be gripped in the user's palm, and the actuating mechanism is toggled between two operative positions, preferably by flexion and extension of a single digit. Alternatively, the handle and actuating mechanism have a plier-like configuration, providing a single instrument adaptable to be held by the user in either of a normal position, with the operative tip directed generally away from the user's body, or a “suicide” position, with the operative tip directed generally toward the user's body.

### FIELD OF THE INVENTION

The present invention relates to endoscopic surgical methods and, in particular, to methods and apparatus for manipulation of suture during endoscopic surgical procedures.

### BACKGROUND OF THE INVENTION

A variety of devices and methods for grasping free suture or passing free suture through tissue during endoscopic surgical procedures have been developed. A hand instrument for retrieving suture within a patient is known from U.S. Pat. Nos. 6,074,403, issued Jun. 13, 2000 to Nord, and U.S. Pat. No. 6,517,552, issued Feb. 11, 2003 to Nord et al. An example of a prior art hand instrument is illustrated in FIG. 9.

Referring to FIG. 9, a suture retriever includes a shaft 1 having a sharp distal tip 2. An opening 3 is formed in the shaft 2. A hinged jaw 4, shown in the closed position in FIG. 9, covers the opening 3. Shaft 1 is provided with a 15 degree upward bend 5. The jaw 4 is operated by a hand mechanism that includes a stationary thumb loop 6 and a moveable finger loop 7 secured by pivot 8. Moveable finger loop 7 actuates closing and opening of jaw 4 by way of a direct connection between the actuating mechanism and the jaw. A camlock mechanism operated by lever 9 locks the jaw in a closed position, for example, to facilitate insertion of the suture retriever through tissue.

Since the introduction of the instruments set forth in the above-noted patent, it has been found that most surgeons do not actually utilize the finger loops during surgery, but instead prefer to activate the instrument jaw by simply pushing on the outside of the moveable finger loop. Accordingly, it would be desirable to provide an instrument with a simplified actuating mechanism which facilitates use of the instrument during surgery.

### SUMMARY OF THE INVENTION

The present invention provides an articulating instrument for retrieving suture within a patient. The articulating suture instrument includes a handle and a shaft having a proximal end and a distal end. An opening in the shaft is disposed proximal to the distal end. A hinged jaw disposed on the shaft closes to capture suture within the opening in the shaft. The motion of an actuating mechanism including a mechanical lever located on the instrument handle is translated through a connecting rod to urge the hinged jaw of the articulating suture instrument into “open” or “closed” positions.

The lever is easily accessed and operated by a single digit while the handle is held in the user's hand. According to an exemplary embodiment, the handle extends longitudinally from the shaft and is held or gripped in the user's palm. The lever toggles readily between two opposite positions through manipulation by the finger or thumb of the hand in which the instrument handle is held or gripped. In an alternative embodiment, the handle and lever are configured in a plier-type arrangement, the handle extending longitudinally from the shaft.

When the jaw of the articulating suture instrument is closed, suture captured within the opening by the jaw is allowed to slide freely as the suture instrument passes through soft tissue. As the instrument is retracted, the captured suture is pulled back through the tissue, the suture being allowed to slide freely through the closed opening to prevent damage to the suture. Preferably, the jaw is locked in the closed position.

Other features and advantages of the present invention will become apparent from the following description of the invention which refers to the accompanying drawings.

### BRIEF DESCRIPTION OF THE DRAWINGS

FIG. 1 illustrates a perspective view of an articulating suture retriever according to the present invention;

FIG. 2 illustrates a partial cross-sectional view of the actuating mechanism of the articulating suture retriever of FIG. 1;

FIG. 3 illustrates an elevation view of the articulating suture retriever of FIG. 1 shown in the open position;

FIG. 4 illustrates an elevation view of the articulating suture retriever of FIG. 1 shown in the closed position;

FIG. 5 illustrates an elevation view of an articulating suture retriever according to an alternative embodiment of the present invention;

FIG. 6 illustrates a partial cross-sectional view of the actuating mechanism of the articulating suture retriever of FIG. 5;

FIG. 7 is a perspective view of an articulating suture retriever according to a second alternative embodiment of the present invention;

FIG. 8 is a perspective view of an articulating suture retriever according to a third alternative embodiment of the present invention; and

FIG. 9 illustrates a prior art articulating suture retriever.

### DETAILED DESCRIPTION OF THE PREFERRED EMBODIMENTS

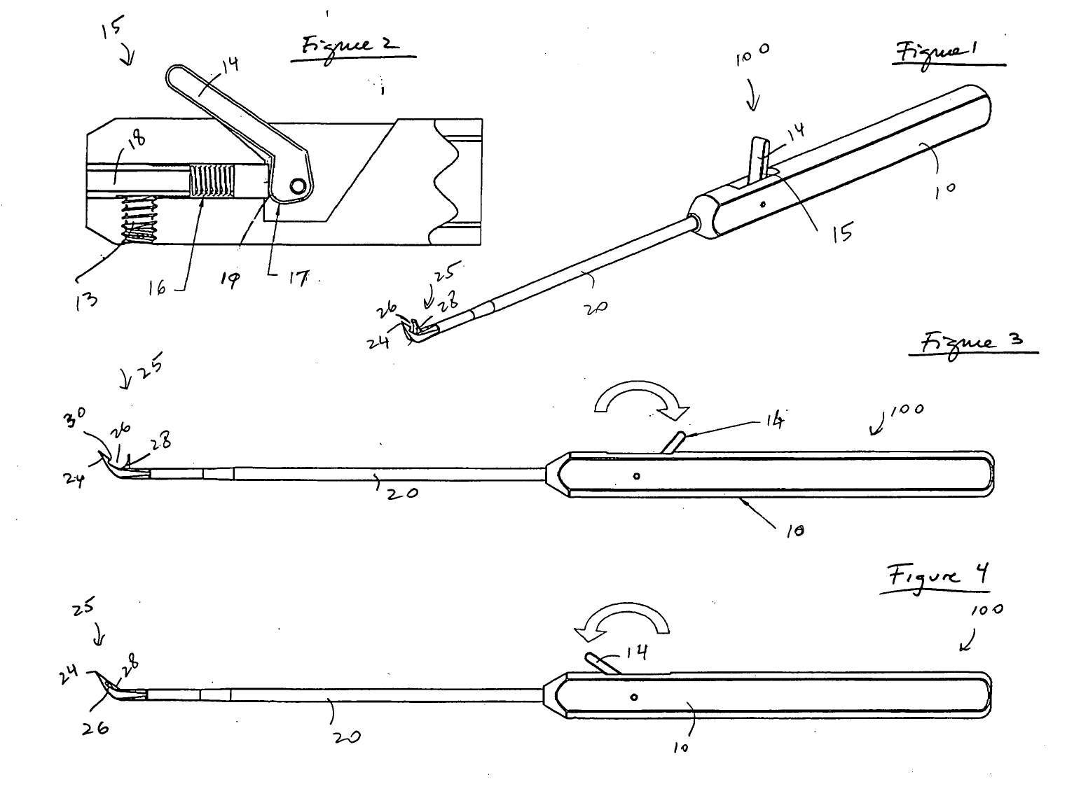

Referring to the drawings, where like elements are designated by like reference numerals, FIGS. 1-4 illustrate an exemplary embodiment of an articulating suture retriever 100 of the present invention. The articulating suture retriever 100 of FIGS. 1-4 may be employed, for example, to manipulate and retrieve suture during arthroscopic surgery.

As illustrated in FIG. 1, the articulating suture retriever 100 includes a handle 10, an actuating mechanism 15, a shaft 20 and a distal tip 25 located at the distal end of the suture retriever. The actuating mechanism 15 is located on handle 10 and is mechanically connected to the distal tip 25 and, when actuated, causes hinged jaw 28 of the distal tip 25 to pivotably rotate between an open or closed position.

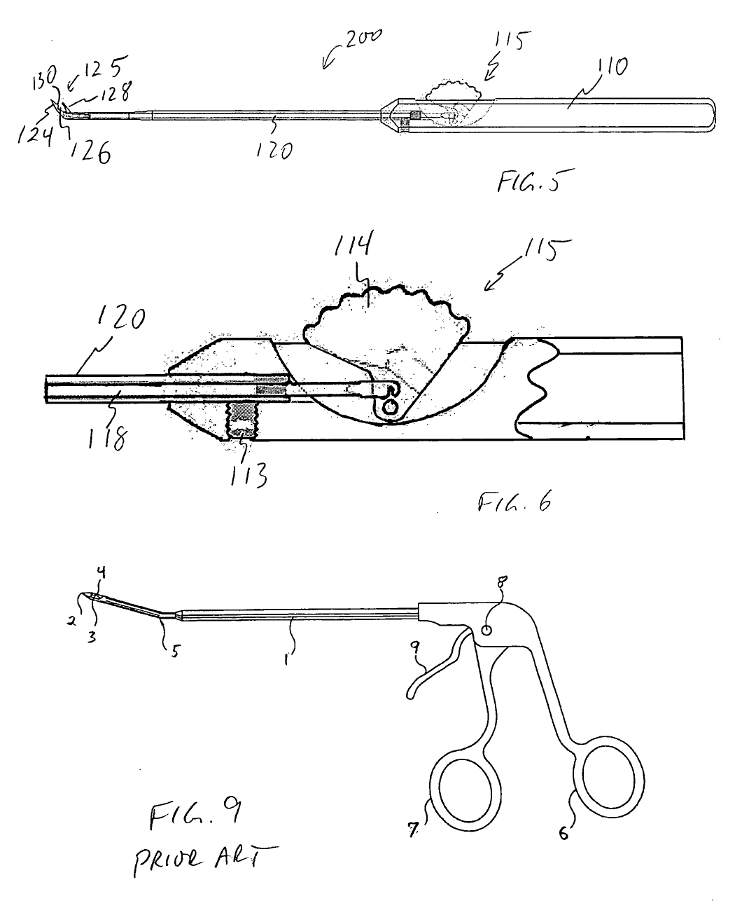

In a preferred embodiment, the distal tip 25 has a structure similar to that of instruments disclosed in U.S. Pat. No. 6,517,552, discussed above. Distal tip 25 features a hinged jaw 28 which covers an opening 26, and terminates in a sharp distal tip 24. In the “closed” position shown in FIG. 4, the outer surface of the hinged jaw 28 forms a smooth surface contiguous with the outer surface of the sharp distal tip 24, meeting the tip at bevel 30. In this manner, a strand of suture captured in the opening 26 when the hinged jaw 28 is closed is free to slide within opening 26 as the articulating suture retriever 100 is pulled through soft tissue, thus protecting the suture from damage. In the “partially-open” and “fully-open” positions of FIGS. 1 and 3, respectively, the hinged jaw 28 opens and provides access to the opening 26 for capturing suture.

FIG. 2 illustrates in more detail the actuating mechanism 15 of the articulating suture retriever 100 of the present invention. The actuating mechanism 15 comprises a mechanical lever or trigger 14 provided with a cam 17, an inner rod or shaft 18, and a spring 16. Shaft 20 of the articulating suture retriever 100 houses the inner rod 18 and is connected to the handle 10. Shaft 20 is held in place by a set screw 13, for example. The inner rod 18 has a proximal end with a radial notch for engaging the hook of trigger 14. Spring 16 is disposed between the proximal end of the inner rod 18 and the proximal end of the shaft 20. Spring 16 forces rod 18 against cam surface 19, as shown in FIG. 2. Trigger 14 is moveable by a flexion or extension of a single digit between a forward position (shown in FIGS. 2 and 4) with the instrument jaw 28 “closed”, a middle position (shown in FIG. 1) with jaw 28 “partially-open”, and a back position (FIG. 3) with jaw 28 “fully-open”. Cam surface 19 is shaped appropriately to allow spring 16 to urge inner rod 18 proximally and thus open the jaw 28 of the instrument when the trigger 14 is moved back. When the trigger is moved forward, the cam surface 19 pivots toward the end of inner rod 19, urge the inner rod 18 distally and thus closing the jaw 28.

FIGS. 3 and 4 are planar views of the articulating suture retriever 100 with the hinged jaw 28 of the distal tip 25 open (FIG. 3) and closed/self-locked (FIG. 4). The hinged jaw 28 of the distal tip 25, which pivotably rotates to open and closed positions, is connected to the actuating mechanism 15 by a springloaded rod 18, which is disposed within shaft 20 of the articulating suture retriever 100. The diameter of rod 18 is smaller than the inner diameter of shaft 20.

FIG. 3 illustrates the trigger 14 of the suture retriever disposed in the back position. Cam surface 19 is designed such that the inner rod moves toward the proximal end of articulating suture retriever 100, whereby spring 16 causes the hinged jaw 28 to rotate to a “fully-open” position.

FIG. 4 illustrates the articulating suture retriever 100 with the trigger 14 pushed forward to the handle stop. When the trigger is moved toward this position, the cam 17 pivots such that lobe 19 urges the inner rod to move toward the distal end of the retriever 100, thus causing the jaw 28 of the distal tip 25 to pivot to the “closed” position. Trigger 14 is locked in the forward position when the cam lobe 19 of the trigger rotates slightly past the center line of the shaft to when pushed forward, binding the mechanism into a locked position. There is no lock at all in the open position. Preferably liver 14 and cam 17 are a unitary construction.

Referring to FIGS. 5 and 6, a suture retriever 200 according to an alternative embodiment of the present invention is shown. Suture retriever 200 includes a handle 110, an actuating mechanism 115, a shaft 120 and a distal tip 125 located at the distal end of the suture retriever. The actuating mechanism 115 is located on handle 110 and is mechanically connected to the distal tip 125 and, when actuated, causes hinged jaw 128 of the distal tip 125 to pivotably rotate between an open or closed position.

In a preferred embodiment, the distal tip 125 has a structure similar to that of instruments disclosed in U.S. Pat. No. 6,517,552, discussed above. Distal tip 125 features a hinged jaw 128 which covers an opening 126, and terminates in a sharp distal tip 124. In the “closed” position, the outer surface of the hinged jaw 128 forms a smooth surface contiguous with the outer surface of the sharp distal tip 124, meeting the tip at bevel 130. In this manner, a strand of suture captured in the opening 126 when the hinged jaw 128 is closed is free to slide within opening 126 as the articulating suture retriever 200 is pulled through soft tissue, thus protecting the suture from damage. In “partially-open” (shown in FIG. 5) and “fully-open” positions, the hinged jaw 128 opens and provides access to the opening 126 for capturing suture.

FIG. 6 illustrates in more detail the actuating mechanism 115 of the articulating suture retriever 200 of the present invention. The actuating mechanism 115 comprises a mechanical thumb lever or trigger 114 connected to an inner rod or shaft 118. Shaft 120 of the articulating suture retriever 200 houses the inner rod 118 and is connected to the handle 110. Shaft 120 is held in place by a set screw 113, for example. The inner rod 118 has a proximal end with a radial notch for engaging trigger 114. Trigger 114 is moveable between a forward position with the instrument jaw 128 “closed”, a middle position with jaw 128 “partially-open”, and a back position with jaw 128 “fully-open”. When the trigger 114 is moved forward, the inner rod 118 is urged distally and thus closes the jaw 128.

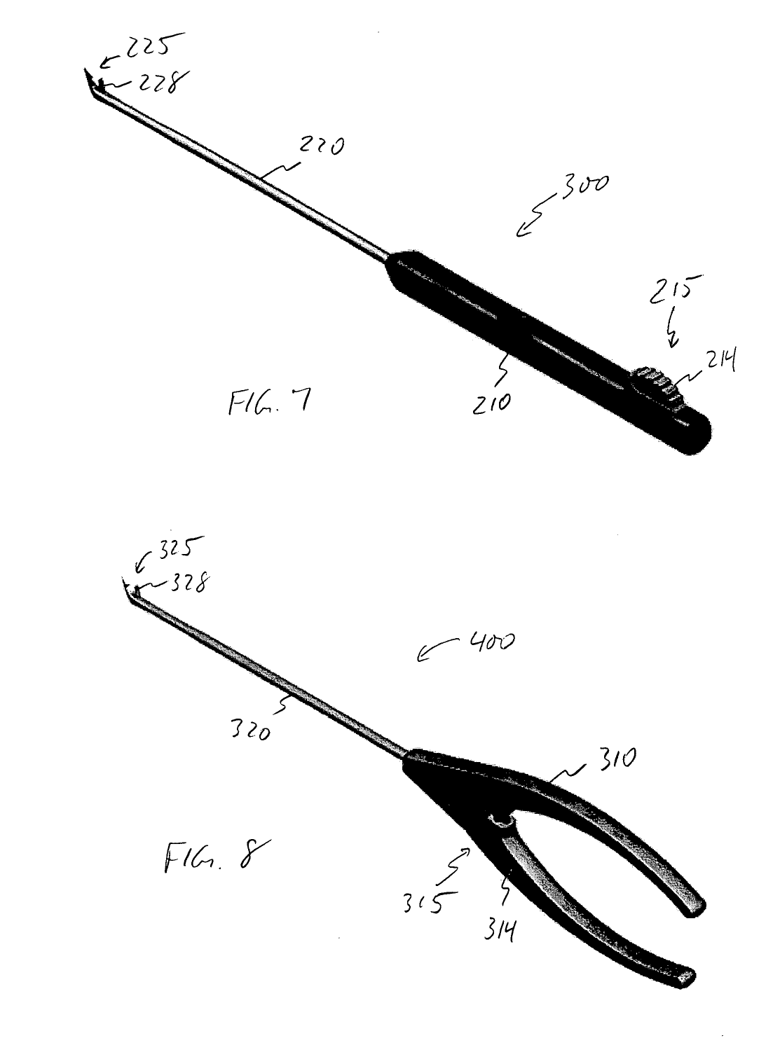

Referring to FIG. 7, a suture retriever 300 according to a second alternative embodiment of the present invention is shown in perspective view. Suture retriever 300 includes a handle 210, an actuating mechanism 215, a shaft 220 and a distal tip 225 located at the distal end of the suture retriever. The actuating mechanism 215 is located proximally on handle 210 and is mechanically connected to the distal tip 225. Actuation of trigger 214 causes hinged jaw 228 of the distal tip 125 to pivotably rotate between an open or closed position. Suture retriever 300 is adapted to be held in like a knife or ice pick in a “suicide” orientation such that the trigger 214 is disposed on a proximal end of the handle away from the shaft 220. The handle 210 of the instrument is held across the palm of a user's hand with the shaft extending distally away from the user's thumb. Accordingly, trigger 214 is positioned for easy manipulation by the user's thumb by flexion or extension.

Referring to FIG. 8, a suture retriever 400 according to a third alternative embodiment of the present invention is shown in perspective view. Suture retriever 400 has a plier-like grip that includes a handle 310, an actuating mechanism 315, a shaft 320 and a distal tip 325 located at the distal end of the suture retriever. Handle 310 is stationary and extends proximally in the longitudinal direction of shaft 320. The actuating mechanism 315 is located distally on handle 310 and is mechanically connected to the distal tip 325. The handle 310 and the trigger 314 are substantially symmetric about a longitudinal axis of the instrument when held in the closed position. Opening the trigger 314 takes it out of axial symmetry with the handle 310. Manipulating trigger 314 of the actuating mechanism 315 causes hinged jaw 328 of the distal tip 325 to pivotably rotate between open and closed positions. A spring arrangement, optionally disposed between the handle and the lever, for example, urges the instrument into a normally open position. The suture retriever 400 is gripped in a user's hand in a manner similar to a pair of pliers. Advantageously, the instrument can be held in the user's palm with the distal tip 325 pointing away from the body, or reversed in the palm with distal tip 325 pointing toward the body (a “suicide” grip), affording the user greater flexibility in certain surgical situations. Moreover, the user can grip the handle and trigger in one hand without the need for a finger ring or a thumb ring on the handle of the instrument.

The above description and drawings illustrate preferred embodiments which achieve the objects, features and advantages of the present invention. Accordingly, it is not intended that the present invention be limited to the illustrated embodiments, but only by the appended claims.

What is claimed as new and desired to be protected by Letters Patent of the United States is:

1. An arthroscopic instrument comprising:
a shaft;
an articulating mechanism disposed on the shaft;
a handle extending from the shaft;
a lever disposed pivotably on the handle and manipulable by a user to toggle between two opposed positions; and
a rod extending along the shaft and linking the articulating mechanism to the lever.

2. The arthroscopic instrument of claim 1, wherein the handle is unitary and extends longitudinally from the shaft.

3. The arthroscopic instrument of claim 2, wherein the handle is adapted to be gripped in a user's palm.

4. The arthroscopic instrument of claim 1, wherein the lever is adapted to be manipulated by a single digit.

5. The arthroscopic instrument of claim 1, wherein the lever is urged by a spring into one of the two opposed positions.

6. The arthroscopic instrument of claim 5, wherein the lever includes a cam engaging the rod.

7. The arthroscopic instrument of claim 1, wherein the lever is disposed on a proximal end of the handle away from the shaft, the instrument being held across the palm of a user's hand with the shaft extending distally away from the user's thumb.

8. The arthroscopic instrument of claim 1, wherein the handle and the trigger form a pliers-like grip.

9. The arthroscopic instrument of claim 8, wherein the handle and the trigger in a closed position are mutually symmetrical with respect to a longitudinal axis of the shaft.

10. The arthroscopic instrument of claim 9, wherein the handle and the trigger in an open position are asymmetrical with respect to the longitudinal axis of the shaft.

11. An arthroscopic instrument comprising:
a shaft;
an articulating mechanism disposed on the shaft;
a multi-member, pliers-like grip, the grip including:
a stationary handle extending from the shaft; and
a movable lever disposed pivotably and manipulable by a user to toggle between two opposed positions; and
a rod extending along the shaft and linking the articulating mechanism to the lever.

12. The arthroscopic instrument of claim 11, wherein the stationary handle is unitary and extends generally in a longitudinal direction from the shaft.

13. The arthroscopic instrument of claim 11, wherein the stationary handle and the movable lever handle are substantially symmetric across a longitudinal axis of the instrument.

14. The arthroscopic instrument of claim 11, wherein the stationary handle and the movable lever are adapted to be gripped without the need for finger or thumb rings.

15. The arthroscopic instrument of claim 11, wherein the stationary handle and the movable lever are urged into a normally open position.

16. The arthroscopic instrument of claim 15, wherein the stationary handle and the movable lever are urged by a spring into the normally open position.
